(19) United States
(12) Patent Application Publication
(10) Pub. No.: US 2003/0028078 A1
(43) Pub. Date: Feb. 6, 2003

(54) In vivo imaging device, system and method

(76) Inventor: Arkady Glukhovsky, Nesher (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 10/208,832

(22) Filed: Aug. 1, 2002

Related U.S. Application Data
(60) Provisional application No. 60/309,181, filed on Aug. 2, 2001.

(51) Int. Cl. A62B 1/04; A61B 1/04

(52) U.S. Cl. 600/109; 348/65

(57) ABSTRACT

A device, system and method for in vivo imaging. A device may include a typically monochrome image sensor, a plurality of illumination sources, each illumination source having different spectral characteristics, and a controller configured for effecting successive illumination of each of the illumination sources within a single image capture cycle. Typically the image sensor is a monochrome sensor. The illumination may be provided by a white light source and a plurality of filters for filtering illumination from the illumination source. A final image may be obtained by processing precursor images created using different spectra or colors.

### PRIOR PROVISIONAL APPLICATION

The present application claims benefit from prior provisional application No. 60/309,181 entitled “IN VIVO IMAGING METHODS AND DEVICES” and filed on Aug. 2, 2001.

### FIELD OF THE INVENTION

The present invention relates to the field of in-vivo imaging.

### BACKGROUND OF THE INVENTION

Devices and methods for performing in-vivo imaging of passages or cavities within a body are known in the art. Such devices may include, inter alia, various endoscopic imaging systems and devices for performing imaging in various internal body cavities.

Reference is now made to FIG. 1 which is a schematic diagram illustrating an example of a prior art autonomous in-vivo imaging device. The device 10A typically includes a capsule-like housing 18 having a wall 18A. The device 10A has an optical window 21 and an imaging system for obtaining images from inside a body cavity or lumen, such as the GI tract. The imaging system may include an illumination unit 23. The illumination unit 23 may include one or more light sources 23A. The one or more light sources 23A may be a white light emitting diode (LED), or any other suitable light source, known in the art. The imaging system of the device 10A includes an imager 24, which acquires the images and an optical system 22 which focuses the images onto the imager 24.

In some configurations when a capsule or tube shaped device is used, the imager 24 may be arranged so that its light sensing surface 28 is perpendicular to the longitudinal axis of the device 40. Other arrangements may be used.

The illumination unit 23 illuminates the inner portions of the body lumen through an optical window 21. Device 10A further includes a transmitter 26 and an antenna 27 for transmitting the video signal of the imager 24, and one or more power sources 25. The power source(s) 25 may be any suitable power sources such as but not limited to silver oxide batteries, lithium batteries, or other electrochemical cells having a high energy density, or the like. The power source(s) 25 may provide power to the electrical elements of the device 10A.

Typically, in the gastrointestinal application, as the device 10A is transported through the gastrointestinal (GI) tract, the imager, such as but not limited to the multi-pixel imager 24 of the device 10A, acquires images (frames) which are processed and transmitted to an external receiver/recorder (not shown) worn by the patient for recording and storage. The recorded data may then be downloaded from the receiver/recorder to a computer or workstation (not shown) for display and analysis.

During the movement of the device 10A through the GI tract, the imager may acquire frames at a fixed or at a variable frame acquisition rate. For example, in one example the imager (such as, but not limited to the imager 24 of FIG. 1) may acquire images at, for example, a fixed rate of two frames per second (2 Hz). However, other different frame rates may also be used, depending, inter alia, on the type and characteristics of the specific imager or camera or sensor array implementation that is used, and on the available transmission bandwidth of the transmitter 26. The downloaded images may be displayed by the workstation by replaying them at a desired frame rate. In this way, the expert or physician examining the data is provided with a movie-like video playback, which may enable the physician to review the passage of the device through the GI tract.

It may generally be desirable to decrease the size and particularly the cross sectional area of in vivo imaging devices, such as the device 10A of FIG. 1, or of imaging devices that are to be inserted into working channels of endoscope-like devices, or integrated into catheter-like devices which may be used in conjunction with guide wires, or the like. Smaller catheter like devices with reduced area may be inserted into narrower body cavities or lumens, such as for example, the coronary arteries, the ureter or urethra, the common bile duct, or the like and may also be easier to insert into working channels of other devices such as endoscopes, laparoscopes, gastroscopes, or the like.

Decreasing the cross-sectional area of such devices may be limited by the cross-sectional area of the imaging sensor, such as for example the imager 24 of FIG. 1. In order to decrease the size and the cross sectional area of the imaging sensor one may need to reduce the pixel size.

In certain imaging sensors, the area of a single pixel cannot be indefinitely reduced in size because the sensitivity of the pixel depends on the amount of light impinging on the pixel which in turn may depend on the pixel area.

One possible approach for reducing the imager area may be to use a smaller number of pixels. This approach may however not be always acceptable, since a reduction in pixel number may result in an unacceptable reduction in image resolution.

Typically, color imaging in imaging sensors may be achieved by using an array of color pixels. For example, in a color image sensor such as the imaging sensor 24 of the device 10A of FIG. 1, there may be three types of pixels in the imager. Each type of pixel may have a special filter layer deposited thereon. Generally, but not necessarily, the filters may be red filters, green filters and blue filters (also known as RGB filters). The use of a combination of pixels having red, green and blue filters is also known in the art as the RGB color imaging method. Other color imaging methods may utilize different color pixel combinations, such as the cyan-yellow-magenta color pixels (CYMK) method.

The pixels with the color filters may be arranged on the surface of the imager in different patterns. For example, one type of color pixel arrangement pattern is known in the art as the Bayer CFA pattern (originally developed by Kodak™). Other color pixel patterns may also be used.

Different color pixel data processing methods are known in the art for computing or interpolating the approximate intensities of the different light colors at each pixel (such as, for computing the approximate intensity of the red and green light at a blue color pixel, mutatis mutandis.) These approximation methods may employ the known intensity of light measured by the color pixel surrounding the pixel for which the calculation is made. For example, the intensity of the blue light at a red color pixel may be approximately computed using the intensity data of the blue color pixels surrounding the red pixel or in the vicinity thereof. Similarly, the intensity of the green light at a red color pixel may be approximately computed using the intensity data of the green color pixels surrounding the red pixel or in the vicinity thereof, mutatis mutandis. These color approximation computations may be typically performed after the pixel data is read out, in a color post-processing computational or processing step, and may depend on the type of color pixel arrangement used in the imaging sensor, as is known in the art.

A problem encountered in the use of RGB color pixel arrays within in-vivo imaging device such as swallowable capsules, or catheter-like devices, or endoscopes, or endoscope like devices, is that for color imaging, one typically needs to use imagers having multiplets of color pixels. Thus, for example in an imager using RGB pixel triplets, each triplet of pixels roughly equals one image pixel because a reading of the intensities of light recorded by the red pixel, the green pixel and the blue pixel are required to generate a single color image pixel. Thus, the image resolution of such a color image may be lower than the image resolution obtainable by a black and white imaging sensor having the same number of pixels. The converse of this is that for a given number of pixels a greater area may be needed for the imager.

Thus, there is a need for an imaging device using a higher resolution and/or smaller area imager.

### BRIEF DESCRIPTION OF THE DRAWINGS

The invention is herein described, by way of example only, with reference to the accompanying drawings, in which like components are designated by like reference numerals, wherein:

FIG. 1 is a schematic diagram illustrating an example of a prior art autonomous in-vivo imaging device;

FIG. 2A is a schematic functional block diagram illustrating an in vivo imaging device, in accordance with an embodiment of the present invention;

FIG. 2B depicts a receiving and a display system according to an embodiment of the present invention;

FIG. 3 is a schematic timing diagram illustrating an exemplary timing schedule which may be usable for performing color imaging in the imaging device illustrated in FIG. 2A;

FIG. 4 is a schematic front view diagram illustrating an exemplary configuration of light sources having different spectral characteristics relative to the optical system of an in vivo imaging device, in accordance with an embodiment of the present invention;

FIG. 5A illustrates a series of steps of a method according to an embodiment of the present invention;

FIG. 5B illustrates a series of steps of a method according to an embodiment of the present invention; and

FIG. 6 illustrates a set of precursor images and a final image according to an embodiment of the present invention.

### SUMMARY OF THE INVENTION

Embodiments of the present invention provide a device, system and method for in vivo imaging. A device according to one embodiment of the invention may include an image sensor, a plurality of illumination sources, each illumination source having different spectral characteristics, and a controller configured for effecting successive (or sequential) illumination of each of the illumination sources within a single image capture cycle. Typically the image sensor is a monochrome sensor.

According to another embodiment a device includes an image sensor, a white light illumination source, a plurality of filters for filtering illumination from the illumination source and a controller configured for effecting successive (or sequential) filtering of the illumination within a single image capture cycle. Typically the image sensor is a monochrome sensor and the filters are red, green or blue filters or any combination thereof.

According to an embodiment of the invention a final image is obtained by processing the precursor images created using different spectra or colors. For example, the precursor images may be combined to produce color images. In one example, a set of images, each created using one of red, green or blue illumination, may be captured and then combined to form a final color image.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Some embodiments of the present invention are based on providing within an in vivo imaging device an imaging pixel array sensor having, typically, a small cross-sectional area using, for example, gray scale imaging pixels without color filters. Such an imaging sensor may be used in conjunction with a plurality of light sources having different spectral characteristics which provide successive or sequential illumination of a site imaged by the image sensor with, for example, light having specific spectral characteristics and bandwidth as disclosed in detail hereinafter. Such a system may allow the same pixel to be used to image more than one color or spectrum, increasing the spatial or other efficiency of the imager. Typically, the illumination provide includes visible light, but other spectra may be used.

Embodiments of such an imaging method may be used for implementing in vivo imaging systems and devices such as, but not limited to, swallowable autonomous in-vivo imaging devices (capsule-like or shaped otherwise), and wired or wireless imaging units which are integrated into endoscope-like devices, catheter-like devices, or any other type of in-vivo imaging device that can be introduced into a body cavity or a lumen contained within a body.

It is noted that while the embodiments of the invention shown hereinbelow are adapted for imaging of the gastrointestinal (GI) tract, the devices, systems and methods disclosed may be adapted for imaging other body cavities or spaces.

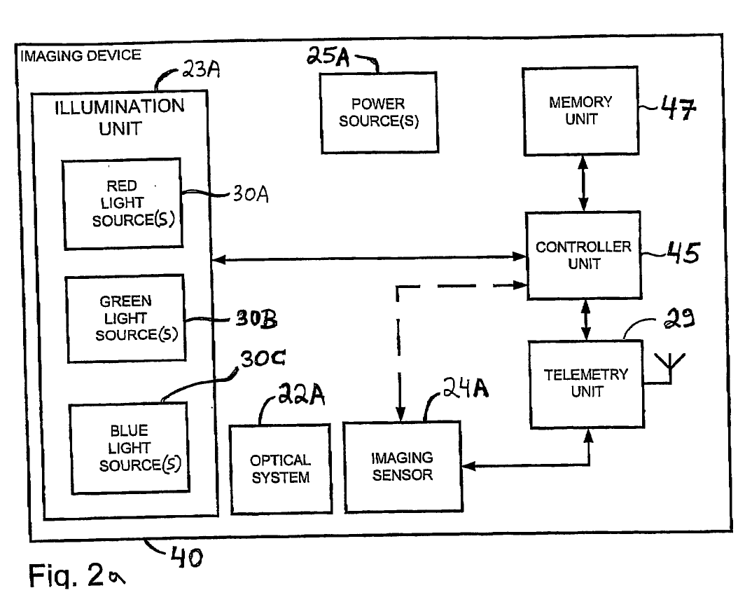

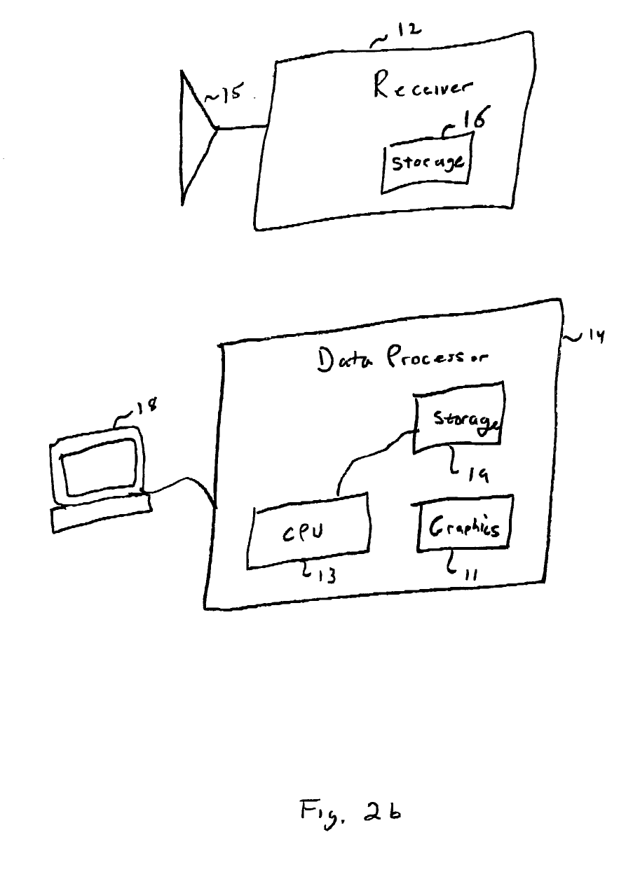

Reference is now made to FIG. 2A which is a schematic functional block diagram illustrating an in vivo imaging device, in accordance with an embodiment of the present invention. In some embodiments, the device and its use are similar to embodiments disclosed in U.S. Pat. No. 5,604,531 to Iddan et al. and/or WO 01/65995 entitled “A Device And System For In Vivo Imaging”, published on Sep. 13, 2001, both of which are hereby incorporated by reference. In other embodiments, other in-vivo imaging devices, receivers and processing units may be used.

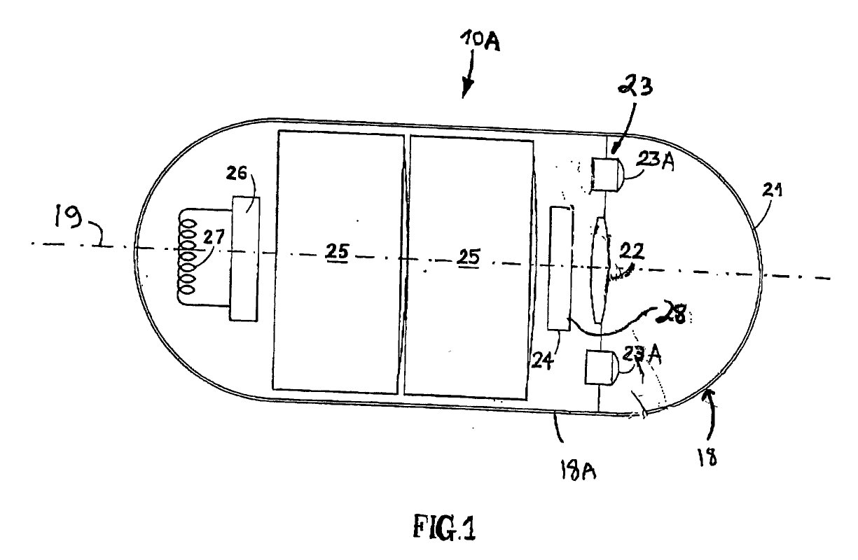

The device 40 may include, for example, an optical system 22A and an imaging sensor 24A. The optical system 22A may be similar to the optical system 22 of FIG. 1 as disclosed hereinabove. The optical system 22A may include one or more optical elements (not shown) which are integrated with the optical system 22A or with another part of device 40, such as for example, a single lens (not shown in FIG. 2) or a compound lens, or any other combination of optical elements, including but not limited to lenses, mirrors, optical filters, prisms or the like, which may be attached to, or mounted on, or fabricated on or adjacent to the light sensitive pixels (not shown) of the imaging sensor 24A.

The imaging sensor 24A may be, for example, a CMOS imaging sensor suitable for gray scale imaging as is known in the art for CMOS sensors having no color filters deposited thereon (except for optional optical filters, such as infrared filters, which may be deposited on the pixels or which may be included in the optical system 22A). Typically the imaging sensor 24A is a monochrome sensor. For example, the CMOS sensor may be capable for producing, in response to being illuminated by light, an output representative of 256 gray levels sensed by each of the pixels (not shown). The number of the pixels in the imaging sensor may vary depending on the specific device or application. For example, the imaging sensor may comprise a 256×256 CMOS pixel array. Other types of sensors which may have different pixel numbers, may however also be used. For example, a CCD may be used.

The device 40 may also include a transmitter or telemetry unit 29 which may be (optionally) suitably connected to the imaging sensor 24A for telemetrically transmitting the images acquired by the imaging sensor 24A to an external receiving device (not shown), such as but not limited to embodiments of the receiver/recorder device disclosed in U.S. Pat. No. 5,604,531 to Iddan et al. The telemetry unit 29 may operate via, for example, radio (RF) waves. The telemetry unit 29 may be constructed and operated similar to the transmitter 26 coupled to the antenna 27 of FIG. 1, but may also be differently constructed and operated as is known in the art for any suitable wireless or wired transmitter. For example, if the device 40 represents an imaging endoscope-like device or catheter-like device, the telemetry unit 29 may be replaced by a wired transmitter (not shown) as is known in the art. In other embodiments, a wired transmitter may be used with devices other than a catheter-like device. In such a case, the wired transmitter may transmit the imaged data to an external workstation (not shown) or processing station (not shown) or display station (not shown), for storage and/or processing and/or display of the image data.

The device 40 may also include a controller unit 45 which may be suitably connected to the imaging sensor 24A for, inter alia, controlling the operation of the imaging sensor 24A. The controller unit 45 may be any suitable type of controller, such as but not limited to, an analog controller, a digital controller such as, for example, a data processor, a microprocessor, a micro-controller, an ASIC, or a digital signal processor (DSP). The controller unit 45 may also comprise hybrid analog/digital circuits as is known in the art. The controller unit 45 may be suitably connected to the telemetry unit 29 and/or other units, for example, for controlling the transmission of image frames by the telemetry unit 29. In alternate embodiments, control may be achieved in other manners. For example, telemetry unit 29 may provide control or act as a controller.

The imaging device 40 typically includes one or more illumination units 23A which may be suitably connected to the controller unit 45. In accordance with one embodiment of the present invention, the illumination units 23A may include one or more red light source(s) 30A, one or more green light source(s) 30B, and one or more blue light source(s) 30C. The red light source(s) 30A, the green light source(s) 30B, and the blue light source(s) 30C may be controlled by the controller unit 45. The red light source(s) 30A as a whole may be considered an illumination unit, and similarly the green light source(s) 30B and blue light source(s) 30C may each be considered illumination units. The red light source(s) 30A, the green light source(s) 30B, and the blue light source(s) 30C may be controlled by the controller unit 45. For example, the controller unit 45 may send to the illumination unit 23A suitable control signals for switching on or off any of the red light source(s) 30A, the green light source(s) 30B, and the blue light source(s) 30C, or subgroups thereof as is disclosed in detail hereinafter.

Other colors and combinations of colors may be used. Typically, the Is illumination provided is visible light and, more specifically, different spectra of visible light, each of which forms a component of a color image. For example, one standard method of providing a color image provides to a viewer red, green and blue pixels, either in spatial proximity or temporal proximity, so that the three color pixels are combined by the viewer to form color pixels. Other sets of visible light forming color images may be used, or visible light not forming color images, and non-visible light may be used. If more than one light source is included within an illumination unit, the light sources may be spread from one another.

The red light source(s) 30A may have spectral characteristics suitable for providing red light which may be used for determining the reflection of light having a wavelength bandwidth within the red region of the spectrum. The spectrum of this red light source may be similar to the spectrum of white light after it was filtered by a typical red filter which may be used in the red pixels of an imager having a typical RGB type pixel triplet arrangement. Other types of red may be used.

Similarly, the green light source(s) 30B may have spectral characteristics suitable for providing green light which may be used for determining the reflection of light having a wavelength bandwidth within the green region of the spectrum. The spectrum of this green light source may be similar to the spectrum of white light after it was filtered by a typical green filter which may be used in the green pixels of an imager having a typical RGB type pixel triplet arrangement. Other types of green may be used.

Similarly, the blue light source(s) 30C may have spectral characteristics suitable for providing blue light which may be used for determining the reflection of light having a wavelength bandwidth within the blue region of the spectrum. The spectrum of this blue light source may be similar to the spectrum of white light after it was filtered by a typical blue filter which may be used in the blue pixels of an imager having a typical RGB type pixel triplet arrangement. Other types of blue may be used.

The exact spectral distribution of the red, green and blue light sources 30A, 30B, and 30C, respectively, may depend, inter alia, on the type and configuration of the light sources 30A, 30B, and 30C.

The light sources 30A, 30B, and 30C may be implemented differently in different embodiments of the present invention. Examples of usable light sources may include but are not limited to, light emitting diodes (LEDs) having suitable (e.g., red, green and blue) spectral characteristics, or other light sources capable of producing white light or approximately white spectral characteristics which may be optically coupled to suitable (e.g., red, green or blue) filters, to provide filtered light having the desired spectral output in the, e.g., red, green or blue parts of the spectrum, respectively. Thus, for example, a blue light source may comprise a white or approximately white light source (not shown) and a suitable blue filter. Green and red light sources may similarly include a white or approximately white light source, optically coupled to suitable green or red filter, respectively.

Such white or approximately white light sources may include LEDs, incandescent light sources, such as tungsten filament light sources, gas discharge lamps or flash lamps, such as for example small xenon flash lamps or arc (amps, or gas discharge lamps, or any other suitable white or approximately white light sources having a suitable spectral range which are known in the art.

The choice of the exact spectral characteristics of the light sources 30A, 30B and 30C, may depend, inter alia, on the sensitivity to different wavelengths of the imaging sensor 24A, on the application, or on other requirements.

The controller unit 45 may be (optionally) suitably connected to the imaging sensor 24A for sending control signals thereto. The controller unit 45 may thus (optionally) control the transmission of image data from the imaging sensor 24A to the telemetry unit 24 (or to a wired transmitter in the case of an endoscopic device, catheter-like device, or the like).

The device 40 may also include a memory unit 47. The memory unit 47 may include one or more memory devices, such as but not limited to random access memory (RAM) units, or other suitable types of memory units known in the art. The memory unit 47 may be used to store the image data read out from the imaging sensor 24A as disclosed in detail hereinafter.

The device 40 may also include one or more power sources 25A. The power source(s) 25A may be used for supplying electrical power to the various power requiring components or circuitry included in the device 40. It is noted that the electrical connections of the power source(s) 25A with the various components of the device 40 are not shown (for the sake of clarity of illustrations). For example, the power source(s) 25A may be suitably connected to the controller 45, the telemetry unit 29, the imaging sensor 24A, the memory unit 47, and the illumination units 23A.

Typically, for autonomous in vivo imaging devices, such as, but not limited to, the swallowable capsule-like device 10A of FIG. 1, the power sources may be batteries or electrochemical cells, as described for the power sources 25 of FIG. 1 The power source(s) 25A may also be any other type of suitable power source known in the art that may be suitably included within the device 40.

The power source(s) 25A may be any other suitable power source such as, for example, a mains operated direct current (DC) power supply or a mains operated alternating current (AC) power supply, or any other suitable source of electrical power. For example, a mains operated DC power supply or a mains operated AC power supply may be used in a device 40 that may be implemented in an endoscope or catheter like device.

Typically, the device 40 is swallowed by a patient and traverses a patient's GI tract, however, other body lumens or cavities may be imaged or examined. The device 40 transmits image and possibly other data to components located outside the patient's body which receive and process the data. FIG. 2B depicts a receiving and a display system according to an embodiment of the present invention. Typically, located outside the patient's body in one or more locations, are a receiver 12, typically including an antenna 15 or antenna array, for receiving image and possibly other data from device 40, a receiver storage unit 16, for storing image and other data, a data processor 14, a data processor storage unit 19, a graphics unit 11, and an image monitor 18, for displaying, inter alia, the images transmitted by the device 40 and recorded by the receiver 12. Typically, the receiver 12 and receiver storage unit 16 are small and portable, and are worn on the patient's body during recording of the images.

Typically, data processor 14, data processor storage unit 19 and monitor 18 are part of a personal computer or workstation which includes standard components such as a processor 13, a memory (e.g., storage 19, or other memory), a disk drive, and input-output devices, although alternate configurations are possible. Typically, in operation, image data is transferred to the data processor 14, which, in conjunction with processor 13 and software, stores, possibly processes, and displays the image data on monitor 18. Graphics unit 11 may, inter alia, form color images from discrete frames of monochrome data, and may perform other functions. Graphics unit 11 may be implemented in hardware or, for example, in software, using processor 13 and software. Graphics unit 11 need not be included, and may be implemented in other manners.

In alternate embodiments, the data reception and storage components may be of another configuration, and other systems and methods of storing and/or displaying collected image data may be used. Further, image and other data may be received in other manners, by other sets of components.

Typically, the device 40 transmits image information in discrete portions. Each portion typically corresponds to a precursor image or frame which is typically imaged using one colored light source spectrum, rather than a broad white spectrum. For example, the device 40 may capture a precursor image once every half second, and, after capturing such an image, transmit the image to the receiving antenna. Other capture rates are possible. Other transmission methods are possible. For example, a series of frames of data recorded with different colors (e.g., R, G, B) may be recorded by the capsule and sent in sequence or as one data unit. In a further embodiment, different frames recorded with different colors may be combined by the capsule and transmitted as one image. Typically, the image data recorded and transmitted is digital image data, although in alternate embodiments other image formats may be used. In one embodiment, each precursor frame of image data includes 256 rows of 256 pixels each, each pixel including data for brightness, according to known methods. The brightness of the overall pixel may be recorded by, for example, a one byte (i.e., 0-255) brightness value. Other data formats may be used.

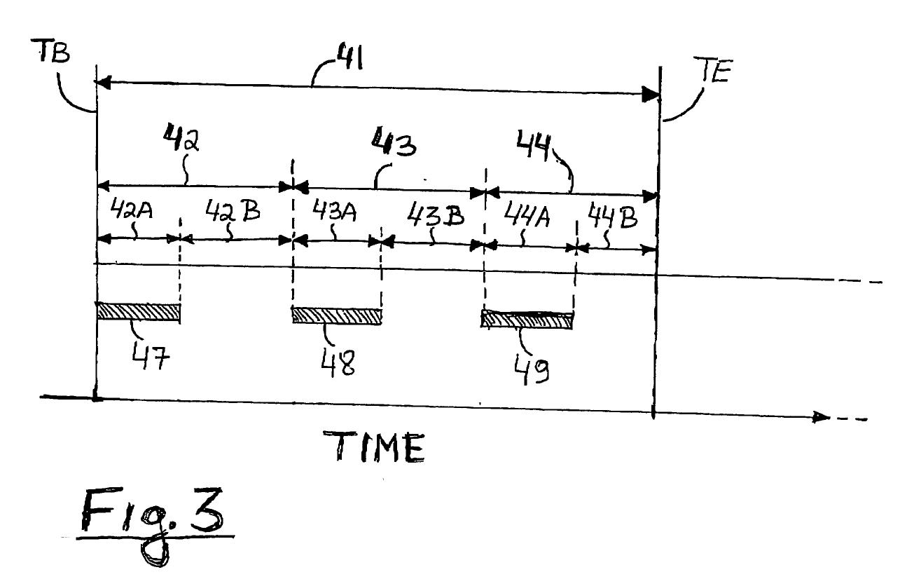

Reference is now made to FIG. 3 which is a schematic timing diagram illustrating an exemplary timing schedule which may be usable for performing color imaging in the imaging device illustrated in FIG. 2A.

The horizontal axis of the graph of FIG. 3 represents time (in arbitrary units). An exemplary imaging cycle 41 (schematically represented by the double headed arrow labeled 41) begins at time TB and ends at time TE. Each imaging cycle may include three different imaging periods 42, 43 and 44. In one embodiment, the imaging cycles are of fixed duration, such as one half second (for two images per second). Other imaging rates may be used, and the imaging cycles need not be of fixed duration. Furthermore, if different numbers of illumination spectra are used, different numbers of imaging periods may be used. For example, an RGBY illumination sequence may require four imaging periods. In alternate embodiment, lights or illumination spectra other than RGB may be used; for example, CMY or other spectra may be used.

Typically, within each period within an imaging cycle, illumination of a certain spectrum or color is provided, and a precursor image is captured using this illumination. Typically, each precursor image captured within an imaging cycle represents substantially the same view of the area to be imaged, as the images are captured within an image capture cycle lasting a relatively short amount of time. For example, given a certain rate of movement, capturing a set of images one half second apart within an image cycle generally results in substantially the same view being imaged in each of the periods. Other rates of imaging may be used, depending on the expected rate of movement. The illumination is provided by a plurality of illumination units, each unit including one or more lights which, as a whole, produce illumination of a certain color. The one or more lights of an illumination unit may be spatially separate. The illumination differs among the periods, to produce images created using different colors reflected to the imager, although, within a cycle, certain colors or spectra may repeat. The order of the colors is typically unimportant, although in some embodiments, the order may be significant.

For example, within the duration of the first imaging period 42 (schematically represented by the double headed arrow labeled 42), imaging is performed using red illumination. For example, the controller unit 45 of the device 40 (FIG. 2A) may switch on or energize the red light source(s) 30A for the duration of the red illumination period 42A (schematically represented by the double headed arrow labeled 42A). The duration of the period in which the red light source(s) 30A provides red light is schematically represented by the hatched bar 47. During the red illumination period 42A, the red light is reflected from (and/or diffused by) the intestinal wall or any other object which is imaged, and part of the reflected and diffused red light may be collected by the optical system 22A (FIG. 2A) and projected on the light sensitive pixels of the imaging sensors 24A. The pixels of the imaging sensor 24A are exposed to the projected red light to produce a precursor image.

After the red illumination period 42A is terminated (by switching off of the red light source(s) 30A by the controller unit 45), the pixels of the imaging sensor 24A may be read out or scanned and transmitted by the telemetry unit 29 to an external receiver/recorder (not shown), or may be stored in the memory unit 47. Thus, a first image is acquired (and may be stored) which was obtained under red illumination. The pixel scanning may be performed within the duration of a first readout period 42B (schematically represented by the double headed arrow labeled 42B).

After the first readout period 42B ends, a second imaging period 43 may begin. Within the duration of the second imaging period 43 (schematically represented by the double headed arrow labeled 43), imaging is performed using green illumination. For example, the controller unit 45 of the device 40 (FIG. 2A) may switch on or energize the green light source(s) 30B for the duration of the green illumination period 43A (schematically represented by the double headed arrow labeled 43A). The duration of the period in which the green light source(s) 30B provide green light is schematically represented by the hatched bar 48. During the green illumination period 43A, the green light is reflected from (and/or diffused by) the intestinal wall or any other object which is imaged and part of the reflected and diffused green light may be collected by the optical system 22A (FIG. 2A) and projected on the light sensitive pixels of the imaging sensors 24A. The pixels of the imaging sensor 24A are exposed to the projected green light.

After the green illumination period 43A is terminated (by switching off of the green light source(s) 30B by the controller unit 45), the pixels of the imaging sensor 24A may be read out or scanned and transmitted by the telemetry unit 29 to an external receiver/recorder (not shown), or may be stored in the memory unit 47. Thus, a second image is acquired (and may be stored) which was obtained under green illumination. The pixel scanning may be performed within the duration of a second readout period 43B (schematically represented by the double headed arrow labeled 43B).

After the second readout period 43B ends, a third imaging period 44 may begin. Within the duration of the third imaging period 44 (schematically represented by the double headed arrow labeled 44), imaging is performed using blue illumination. For example, the controller unit 45 of the device 40 (FIG. 2A) may switch on or energize the blue light source(s) 30C for the duration of a blue illumination period 44A (schematically represented by the double headed arrow labeled 44A). The duration of the period, in which the blue light source(s) 30C provide blue light is schematically represented by the hatched bar 49. During the blue illumination period 44A, the blue light is reflected from (and/or diffused by) the intestinal wall or any other object which is imaged and part of the reflected and diffused blue light may be collected by the optical system 22A (FIG. 2A) and projected on the light sensitive pixels of the imaging sensors 24A. The pixels of the imaging sensor 24A are exposed to the projected blue light.

After the blue illumination period 44A is terminated (by switching off of the blue light source(s) 30B by the controller unit 45), the pixels of the imaging sensor 24A may be read out or scanned and transmitted by the telemetry unit 29 to an external receiver/ recorder (not shown), or may be stored in the memory unit 47. Thus, a third image is acquired (and may be stored) which was obtained under blue illumination. The pixel scanning may be performed within the duration of a third readout period 44B (schematically represented by the double headed arrow labeled 44B).

After the ending time TB of the first imaging cycle 41, a new imaging cycle (not shown) may begin as is disclosed for the imaging cycle 41 (by repeating the same imaging sequence used for the imaging cycle 41). In alternate embodiments illumination from more than one illumination unit may be used per image. For example, while capturing an image, both a set of blue lights (wherein set may include one light) and a set of yellow lights may be used. Within an imaging cycle, certain illumination periods may use the same or substantially the same spectrum of light. For example, there may be two “blue” illumination periods.

It is noted that if the device 40 does not include a memory unit 47, the time periods 42B, 43B, and 44B may be used for transmitting the acquired “red” “green” and “blue” images to the external receiver/recorder or to the processing workstation.

Alternatively, if the image data of the “red” “green” and “blue” images acquired within the duration of an imaging cycle (such as the imaging cycle 41, or the like) are stored within the memory unit 47, the stored “red” “green” and “blue” images of the imaging cycle may be telemetrically transmitted to a receiver/recorder or processing workstation after the imaging cycle 41 is ended.

After the (e.g., red, green and blue) acquired images have been transferred from the receiver/recorder to a processing and display workstation (or after the three images have been transmitted by wire to such a processing and display workstation, for example, in the case of endoscope-like or catheter-like device, or the like), the images data may be processed to produce the final color image for display. Since, typically, each of the different precursor images captured within an imaging cycle represents substantially the same view, combining these images produces that view, but with different color characteristics (e.g., a color image as opposed to monochrome). This may be performed by suitably processing the values of the (gray scale) light intensity data of the same pixel in the, e.g., three (“red”, “green”, and “blue”) imaged data sets. Typically, each monochrome precursor image includes grayscale levels which correspond to one color or spectrum (e.g., levels of red).

For example, each corresponding pixel (typically a monochrome pixel) in a set of images collected using various colors may be combined to produce color pixels, where each resulting color pixel is represented or formed by a set of color levels (e.g., RGB levels) and may include sub-pixels (e.g., one color pixel including RGB sub-pixels). Pixels may be repeatedly combined the within the set of precursor images to produce a final image. In one type of typical monitor, each color pixel is represented by a set of monochrome (e.g., RGB) pixels, and/or by a set of color levels (e.g. RGB color levels).

Other methods may be used. This computation may be corrected (e.g., brightness levels) to account for different sensitivity of the imaging sensor 24A to different wavelengths of light as is known in the art, and other processing, correction or filtering may be performed.

In one embodiment, data processor 14 (FIG. 2B) combines each frame of the series of sets of image frames to produce a series of color images for display monitor 18 or for storage or transmission. The image data is read out from data processor storage unit 19 and processed by, for example, graphics unit 11. Various methods may be used to combine the color image data, and a separate graphics unit need not be used. Other systems and methods of storing and/or displaying collected image data may be used.

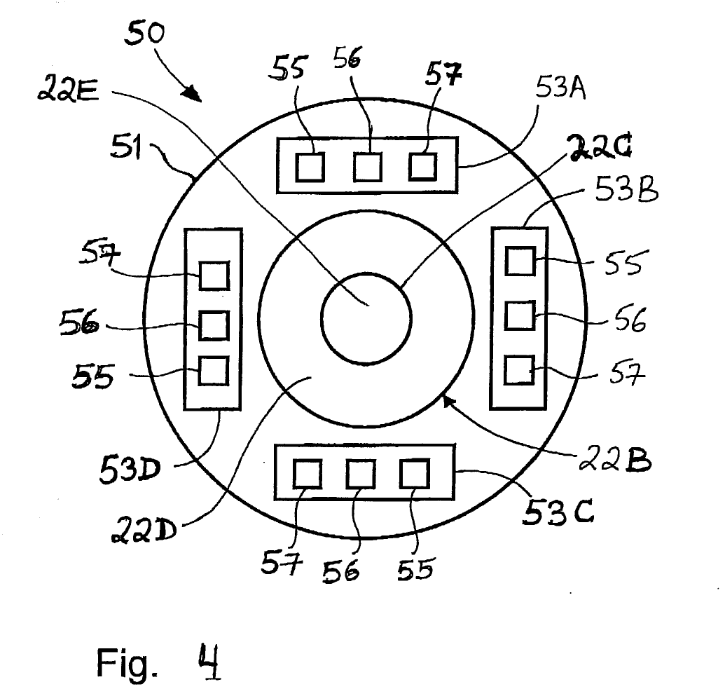

Reference is now made to FIG. 4 which is a schematic front view diagram illustrating an exemplary configuration of light sources having different spectral characteristics relative to the optical system of an in vivo imaging device, in accordance with an embodiment of the present invention.

The device 50 of FIG. 4 is illustrated in front view and may be similar in shape to, for example, the device 1OA of FIG. 1. The optical window 51 of the device 50 may be similar to the dome shaped optical window 21 of the device 10A of FIG. 1; other configurations may be used. The front part of the optical system 22B may include an optical baffle 22D having an opening 22C therethrough. A lens 22E (seen in a frontal view) may be attached within the baffle 22D. Four illumination elements 53A, 53B, 53C, and 53D are arranged attached within the device 50 as illustrated. Typically, the four illumination elements 53A, 53B, 53C and 53D are configured symmetrically with respect to the optical system 22B. In alternate embodiments, other components or arrangements of components may be used. For example, other numbers of illumination elements may be used, and a baffle or other elements may be omitted.

Each of the four illumination elements 53A, 53B, 53C, and 53D includes, for example, a red light source 55, a green light source 56 and a blue light source 57. The light sources 55, 56 and 57 may be LEDs having suitable red, green and blue emission spectra as disclosed hereinabove. Alternatively, the light sources 55, 56 and 57 may be any other suitable compact or small light sources comprising a combination of a white or approximately white light source and a filter having a suitable red, green and blue bandpass characteristics as disclosed hereinabove. Other colors ay be used, and light sources other than LEDs may be used.

The light sources 55, 56 and 57 each may be considered a set of different light units (wherein set may include one), each light unit outputting a different spectrum or color. The spectra used may overlap in whole or in part i.e., in some embodiments, two blue units outputting similar or same blue light may be used, or in some embodiments, two light units outputting different colors may have spectra that overlap to an extent. Each light unit may include one or more lamps. While in the embodiment shown, each light unit includes four spatially separated lamps, other numbers of lamps and patterns may be used.

In operation the device 50 may use a similar illumination schedule as disclosed for the device 40 (an example of one schedule is illustrated in detail in FIG. 3). All the red light sources 55 may be switched on within the duration of the time period 42A (FIG. 3) and terminated at the end of the time period 42A. All the green light sources 56 may be switched on within the duration of the time period 43A (FIG. 3) and terminated at the end of the time period 43A. All the blue light sources 57 may be switched on within the duration of the time period 44A (FIG. 3) and terminated at the end of the time period 44A. The advantage of the light source configuration of the device 50 is that the red, green and blue light sources may distribute the light relatively evenly to achieve relatively uniform light distribution in the field of view of the optical imaging system 22B (FIG. 4). Other configurations may be used, such as configurations not using different banks of colored lights.

It is noted that while the specific light source configuration illustrated in FIG. 4 is suitable for performing an embodiment of the color imaging method of the present invention, many other different light source configurations including different numbers of light sources, different spectra and colors, and different types of light sources may be used. Moreover, the number and the geometrical arrangement of the red, green and blue light sources 55, 56, and 57, respectively, within the four illumination elements 53A, 53B, 53C, and 53D may be different. Furthermore, the number of the illumination elements and their arrangement with respect to the optical system 22B may also be varied.

It is noted that the color imaging method and device disclosed hereinabove need not be limited to methods and devices using RGB illumination sources or the RGB method for color imaging. Other types of color imaging methods may also be used. For example, in accordance with another embodiment of the present invention the light source(s) 30A, 30B, and 30C may be adapted for used with the CYMK method which is well known in the art, by using light sources producing light having cyan, yellow and magenta spectral characteristics as is known in the art. This adaptation may be performed, for example, by using white or approximately white or broadband light sources in combination with suitable cyan, yellow and magenta filters. Other, different spectral color combinations known in the art may also be used.

The use of the CYMK color method may also require proper adaptation of the data processing for color processing and color balancing.

It is noted that the RGB or CYMK illuminating method disclosed hereinabove may have the advantage that they may allow the use of an imaging sensor having a third of the number of color pixels used in a conventional color imager having pixel triplets (such as, but not limited to red, green, and blue pixel triplets or cyan, yellow, and magenta pixel triplets, or the like). In this way, one may reduce the size and the light sensitive area of the imaging sensor without reducing the nominal image resolution.

The use of, for example, the three color illumination periods disclosed hereinabove for an embodiment of the present invention, may provide three temporally separate exposures of the imaging sensor to three different types of colored light, may have other advantages. For example, in the RGB example disclosed hereinabove, the three consecutive exposures of the same pixels to red, green and blue light, the data available after the completion of an imaging cycle includes the intensity of red, green and blue light values which were measured for each pixel of the imaging sensor. It is therefore possible to directly use the measured values for displaying a color image which may simplify the data processing and may improve the resolution and the color quality or fidelity (such as, for example, by reducing color aliasing effects). This is in contrast with other color imaging methods, used in imaging sensors having color pixels arranged in triplets or other arrangement types on the imaging sensor, in which it is necessary to perform the post-processing computations for interpolating or approximating the approximate color intensity at each pixel by using the data of the surrounding pixels having different (or complementary) colors.

It is noted that for autonomous in vivo imaging devices which may be moved through, for example, the GI tract or through other body cavities, caution must be exercised in choosing the duration of the imaging cycle and of the duration of the illumination time periods 42A, 43A and 44A, and the duration of the readout time periods 42B, 43B, and 44B. In some embodiments, the duration of these time periods should be as short as possible to reduce the probability that the device 40 may be moved a substantial distance in the GI tract (or other body cavity or lumen) within the duration of any single imaging cycle. If such a substantial movement of the device 40 occurs within the duration of a single imaging cycle, the “red”, “green” and “blue” acquired images may not be identical (or in other words may not properly register), which may introduce errors in the processing of the final color image which may in turn cause the color image to be blurred or smeared or distorted or not representative of the shape or the true color of the imaged object. Thus, typically, each of the different precursor images captured within an imaging cycle captures substantially the same image or view (e.g., the same view of a portion of an in-vivo area), using a different color or spectrum. Significant movement in between images may result in a different view being imaged. Of course, where movement is less of a problem, timing may be less of an issue.

In insertable devices such as endoscopes or catheter-like devices, the device may be held relatively static with respect to the imaged object, which may allow the use longer duration of the illumination time periods 42A, 43A and 44A, and the duration of the readout time periods 42B, 43B, and 44B.

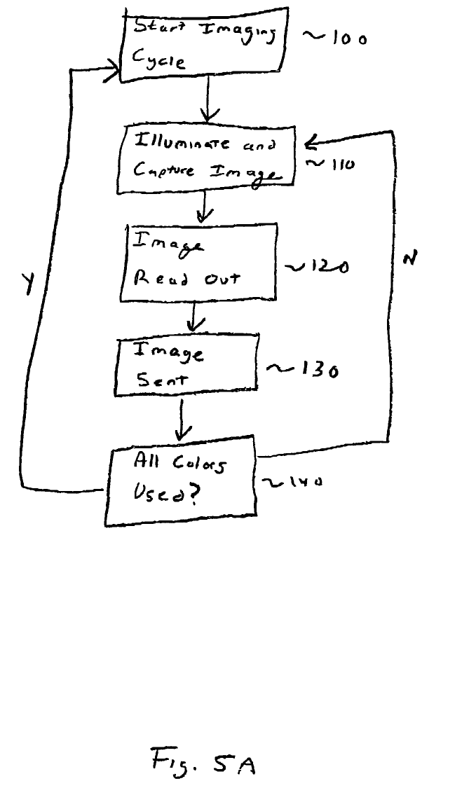

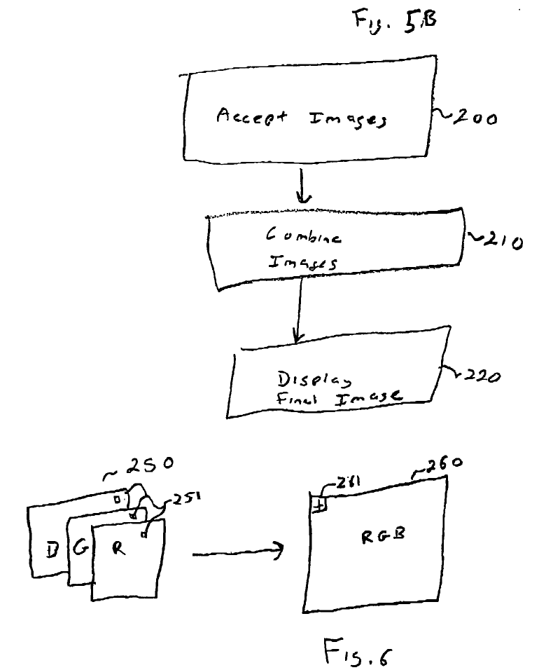

FIG. 5A illustrates a series of steps of a method according to an embodiment of the present invention. Referring to FIG. 5A, in step 100, an imaging cycle starts.

In step 110, a single color or spectrum of light illuminates an area to be imaged, and an image is captured. Typically, step 110 is repeated at least once more with another color or spectrum of light.

In step 120, the image is read out to, for example, a memory device or transmitter. In alternate embodiments, no image readout separate from transmission, processing, or storage need be used.

In step 130, the image is transmitted or otherwise sent to a receiving unit. In an alternate embodiment, the image data may simply be recorded or stored, or the set of images comprising a color image may be sent at the end of an image cycle.

In step 140, if all of the set of colors or spectra have been used to capture an image, the image cycle process starts again at step 100. If further colors or spectra are to be used, the method proceeds to step 110 to image using that color or spectrum.

In alternate embodiments, other steps or series of steps may be used.

FIG. 5B illustrates a series of steps of a method according to an embodiment of the present invention. Referring to FIG. 5B, in step 200, a processor accepts a set of precursor images from, for example, an in vivo imaging device. Typically, each precursor image is a monochrome image created using a non-white light or spectrum, and represents the same view.

In step 210, the set of precursor images is combined to form one final image. For example, referring to FIG. 6, a set of images 250, containing pixels such as pixels 251, may be combined to produce a final image 260, containing composite pixels such as pixel 261. In the example shown in FIG. 6, a set of R, G and B pixels 251, each typically representing substantially the same area of an image, are combined to form one pixel 261, which may include, for example, RGB sub-pixels. Other image formats and other methods of combining images may be used; for example, the final image may include temporal combination of colors.

In step 220, the final image may be displayed to a user. Alternately, the image may be stored or transmitted.

In alternate embodiments, other steps or series of steps may be used.

While the invention has been described with respect to a limited number of embodiments, it will be appreciated that many variations, modifications and other applications of the invention may be made which are within the scope and spirit of the invention.

Embodiments of the present invention may include apparatuses for performing the operations herein. Such apparatuses may be specially constructed for the desired purposes (e.g., a “computer on a chip” or an ASIC), or may comprise general purpose computers selectively activated or reconfigured by a computer program stored in the computers. Such computer programs may be stored in a computer readable storage medium, such as, but is not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions.

The processes presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems appears from the description herein. In addition, embodiments of the present invention are not described with reference to any particular programming language. It will be appreciated that a variety of programming languages may be used to implement the teachings of the invention as described herein.

Unless specifically stated otherwise, as apparent from the discussions herein, it is appreciated that throughout the specification discussions utilizing terms such as ”processing “computing”, “calculating”, “determining”, or the like, typically refer to the action and/or processes of a computer or computing system, or similar electronic computing device (e.g., a “computer on a chip” or ASIC), that manipulate and/or transform data represented as physical, such as electronic, quantities within the computing system's registers and/or memories into other data similarly represented as physical quantities within the computing system's memories, registers or other such information storage, transmission or display devices.

1. An in-vivo imaging device operating over a series of imaging cycles, each cycle including a plurality of imaging periods, the device comprising:
an image sensor;
a set of light units, each light unit outputting a different spectrum of light;
wherein, during each imaging period, at least one light unit provides illumination and the image sensor captures a precursor image and wherein for at least two image periods a different illumination spectrum is provided.

2. The device of claim 1, comprising a controller, the controller capable of controlling the illumination and imaging.

3. The device of claim 1, wherein the device is a swallowable capsule.

4. The device of claim 1, wherein the device is configured to image the GI tract.

5. The device of claim 1, wherein the image sensor includes a CMOS.

6. The device of claim 1, comprising an RF transmitter.

7. The device of claim 1, wherein the transmitter is capable of, after each precursor image is captured, transmitting the precursor image.

8. The device of claim 1, wherein at least one light unit includes red lights.

9. The device of claim 1, wherein at least one light unit includes green lights and at least one light unit includes blue lights.

10. The device of claim 1, wherein each spectrum includes visible light.

11. The device of claim 1 wherein the set of lights include LEDs.

12. The device of claim 1 wherein the set of lights include filters.

13. The device of claim 1 wherein the image sensor is a monochrome sensor.

14. The device of claim 1 wherein the set of precursor images captured during an imaging cycle may be combined to produce a color image.

15. A system capable of receiving images from the device of claim 1, and capable of combining a set of images to produce a color image.

16. A method for performing color imaging in an in-vivo imaging device operating over a set of imaging cycles, the method comprising:
during each of a set of imaging periods within an imaging cycle, providing illumination and capturing a precursor image, wherein, for at least two of the imaging periods, the spectrum of the illumination provided is different.

17. The method of claim 16 wherein the device includes an image sensor.

18. The method of claim 16 wherein the device includes a CMOS imager.

19. The method of claim 16 wherein the device includes a monochrome image sensor.

20. The method of claim 16, wherein the device includes a set of light units, each light unit outputting a different spectrum of light.

21. The method of claim 16, wherein the device is a swallowable capsule.

22. The method of claim 16, comprising imaging the GI tract.

23. The method of claim 16, comprising transmitting the precursor images using RF waves.

24. The method of claim 16, comprising providing red illumination.

25. The method of claim 16, comprising providing green and blue illumination.

26. The method of claim 16, wherein each spectrum includes visible light.

27. The method of claim 16 comprising providing illumination via LEDs.

28. The method of claim 16 comprising passing light through filters.

29. The method of claim 16 comprising combing a set of precursor images captured during an imaging cycle to produce a color image.

30. A system for displaying images, the system comprising:
a controller capable of accepting a plurality of sets of precursor images from an in-vivo device, each of the plurality of sets of precursor images including a plurality of monochrome images, and capable of, for each set of precursor images, combining the precursor images to produce a color image.

31. The system of claim 30, wherein the precursor images are received via radio waves.

32. The system of claim 30, wherein the controller is capable of combining a set of monochrome pixels to produce a color pixel.

33. The system of claim 30, wherein the controller is capable of combining a set of color levels to produce a color pixel.

34. The system of claim 30, wherein each precursor image within a set of precursor images represents substantially the same view.

35. A system for displaying images, the system comprising:
a controller means for accepting a plurality of sets of precursor images from an in-vivo device, each of the plurality of sets of precursor images including a plurality of monochrome images, and, for each set of images, combining the precursor images to produce a color image.

36. A system for displaying images, the system comprising:
a controller capable of accepting a plurality of sets of precursor images from an in-vivo device, each precursor image within a set of precursor images representing substantially the same view, each of the plurality of sets of precursor images including a plurality of monochrome images, and capable of, for each set of precursor images, repeatedly combining a set of monochrome pixels to produce a color pixel, so that the set of precursor images is combined to produce a color image.

37. A method for displaying images, the method comprising:
accepting a plurality of sets of precursor images from an in-vivo device, each of the plurality of sets of precursor images including a plurality of monochrome images; and
for each set of precursor images, combining the precursor images to produce a color image.

38. The method of claim 37, wherein the precursor images are received via radio waves.

39. The method of claim 37, comprising combining a set of monochrome pixels to produce a color pixel.

40. The method of claim 37, comprising combining a set of color levels to produce a color pixel.

41. The method of claim 37, wherein each precursor image within a set of precursor images represents substantially the same view.

42. A method for displaying images, the method comprising:
accepting a plurality of sets of precursor images from an in-vivo device, each of the plurality of sets of precursor images including a plurality of monochrome images, wherein each precursor image within a set of precursor images represents substantially the same view; and
for each set of precursor images, combining the pixels of the precursor images to produce a color image.

43. A swallowable in-vivo imaging capsule comprising:
a CMOS imager;
a plurality of light units, each light unit outputting a different color of light;
wherein, during each of a plurality of imaging periods, at least one light unit is capable of providing illumination and the image sensor is capable of capturing a precursor image and wherein for at least two image periods a different illumination color is provided.

44. An in-vivo imaging unit operating over a plurality of imaging cycles, each cycle including a set of imaging periods, the imaging unit comprising:
an image sensor;
a controller;
an RF transmitter;
a plurality of light units, each light unit outputting a different spectrum;
wherein, during each of a plurality of imaging periods, at least one light unit provides illumination and the image sensor captures an image and wherein for at least two image periods a different spectrum is provided.

45. An in-vivo imaging unit comprising:
an image sensor means for capturing an image;
a controller means for controlling the operation of the capsule;
a plurality of light unit means, each light unit means for outputting a different spectrum;
wherein, during each of a plurality of imaging periods, at least one light unit means provides illumination and the image sensor means captures an image and wherein for at least two image periods a different spectrum is provided.

46. A method for performing color imaging in a swallowable in-vivo imaging capsule operating over a plurality of imaging cycles, the method comprising:
during each of a plurality of imaging periods within an imaging cycle, providing illumination and capturing a precursor image, wherein, for at least two of the imaging periods, the spectrum of the illumination provided is different; and
after each imaging period, transmitting the precursor images.

47. A method for performing imaging in an in-vivo imaging unit operating over a plurality of imaging cycles, the method comprising:
during each of a plurality of imaging periods within an imaging cycle, providing illumination via LEDs and capturing an image, wherein, for at least two of the imaging periods, the color of the illumination provided is different; and
transmitting the images via radio waves.
